SEQUENCE LISTING (4 sequences)

SEQ ID NO: 1 - DNA, 36 bp, Artificial
  Gene-specific primer containing gene-specific overlap region plus portion of OLE1 starter sequence
ccaaaggatgactctgccagcagtggcattgtcgac

SEQ ID NO: 2 - DNA, 60 bp, Artificial
  Forward primer containing remainder of OLE1 starter sequence
ccaacttctggaactactattgaattgattgacgaccaatttccaaaggatgactctgcc

SEQ ID NO: 3 - DNA, 59 bp, Artificial
  Forward primer containing ribosomal binding site and recombination site for Gatewayr cloning.
ggggacaagtttgtacaaagcaggctccaataatgtctccaacttctggaactactatt

SEQ ID NO: 4 - DNA, 30 bp, Artificial
  Reverse primer containing recombination site for Gatewayr cloning.
ggggaccactttgtacaagaaagctgggtc

(19) United States
(12) Patent Application Publication
(10) Pub. No.: US 2008/0182249 A1
(43) Pub. Date: Jul. 31, 2008

(54) Expression systems for mammalian and mycobacterial desaturases

(76) Inventors: Brian G. Fox, Madison, WI (US); Pablo Sobrado, Blacksburg, VA (US); Yong Chang, Madison, WI (US)

Correspondence Address: BRINKS HOFER GILSON & LIONE, P.O. BOX 10395, CHICAGO, IL, 60610, US

(21) Appl. No.: 11/899,081

(22) Filed: Sep. 4, 2007

Related U.S. Application Data
(60) Provisional application No. 60/841,825, filed on Sep. 1, 2006.

(51) Int. Cl. C12Q 1/68; C12N 15/63

(52) U.S. Cl. 435/6; 435/320.1

(57) ABSTRACT

Expression system for components of a desaturase complex is provided. The system includes expression of a desaturase and an oxidoreductase. The system may be used for expression of mycobacterial desaturases or for expression of mammalian desaturases. The system may further include cell-free expression of other components of the desaturase complex. The expression system may include expression of stearoyl-CoA. The expression system may further include expression of cytochrome b5. The expression system may also include expression of cytochrome b5 reductase. The expression system may also include expression of Rv3230c. In addition, methods for assaying the activity of a stearoyl-CoA desaturase in vitro are provided.

### CROSS-REFERENCE TO RELATED APPLICATIONS

This invention claims priority to U.S. Provisional Patent Application Ser. No. 60/841,825 filed on Sep. 1, 2006, which is incorporated herein by reference.

### GOVERNMENT INTERESTS

This invention was made with United States government support from the National Institutes of Health (NIH), grant number GM050853. The United States government may have certain rights in this invention.

### FIELD OF THE INVENTION

This invention is related to the biomedical arts. The present invention provides an expression system that allows for the characterization of enzymes involved in the synthesis of unsaturated fatty acids.

### BACKGROUND OF THE INVENTION

The integral membrane desaturases are an enzyme family of immense biomedical and industrial importance. The significance of the desaturases arises from their fundamental contributions to lipid compositions and cellular homeostasis. In both eukaryotes and prokaryotes, desaturases produce essential mono- and polyunsaturated precursors to the lipid components of all cell membranes and thus help to control and maintain membrane function. Therefore, desaturases may be involved in human diseases associated with changes in lipid composition, including obesity, diabetes, hypertension, cardiovascular disease, immune disorders, degenerative neurological diseases, and skin diseases. Links between monounsaturated fatty acids and the regulation of apoptosis, neuronal differentiation, and signal transduction have been reported. The influence of monounsaturated fatty acids on apoptosis may be coupled to the development of some tumors (Lu et al., 1997, J. Mol. Carcinog. 20: 204-215; Falvella et al., 2002, Carcinog. 11: 1922-1936). Also, the fatty acid composition of erythrocyte membranes is associated with breast cancer risk (Pala et al., 2001, J. Nat. Canc. Inst. 93: 1088-1095).

Stearoyl-CoA desaturase (SCD) catalyzes the rate-determining step in the synthesis of monounsaturated fatty acids. SCD introduces a double bond between positions 9 and 10 of stearoyl-CoA (18:0) and palmitoyl-CoA (16:0). The activity of SCD influences the fatty acid composition of membrane phospholipids, triglycerides, and cholesterol esters. Alterations of SCD activity result in changes of membrane fluidity, lipid metabolism, and metabolic rate.

Transgenic mice (Mus musculus) with a mutation in stearoyl-CoA desaturase 1 (SCD1) have increased energy expenditure, reduced body adiposity, and remain lean when subject to a high calorie diet, despite a higher food intake as compared to control mice (Ntambi et al., 2003, Prog. Lipid. Res. 43: 91-104). These findings, limited to analysis of the SCD function, link SCD function to a major health epidemic, obesity, and identify SCD as potential target for anti-obesity drugs.

Unsaturated fatty acids are also precursors of mycolic acid, a wax-like coating that protects human pathogens such as Mycobacterium tuberculosis from desiccation, macrophage attack, water-soluble antibiotics, and other ameliorative agents. Desaturases are of great importance to insects in the biosynthetic pathways for production of juvenile maturation hormones, and in the use of fatty acids as an energy source during swarming. Desaturases also contribute to the composition of all plant seed oils consumed by humans, and are recognized as relevant enzymes for renewable sources of hydrocarbons.

Each of the above areas involving desaturases has high impact on human health or areas of economic interest. It is therefore important to improve our understanding of the mechanisms in which fatty acid desaturation proteins function, and to understand the consequence of these enzymatic reactions on cellular structure and function.

The desaturase enzyme family is defined by the Pfam database (Bateman et al., 2004, Nucl. Acids Res. 32: D138-D141). SCD from yeast, rat, and mice are each members of the class III diiron family of enzymes. The hallmark of the membrane-bound SCD enzymes is that all contain an eight histidine motif (HX(₃₋₄)H— —HX(₂₋₃)HH— —HX(₂₋₃)HH). Site-directed mutagenesis in rat SCD has demonstrated that all eight histidines are essential for activity and it was postulated that at least some of these residues were necessary for binding the iron atoms. Four isoforms of SCD have been identified in mice (SCD1-4). SCD1 is expressed largely in the liver and adipose tissue. SCD2 is expressed in the mouse brain, heart, lungs, kidney, spleen, and adipose tissue. SCD3 is expressed in the skin, Harderian gland, and preputial gland. SCD4 is expressed exclusively in the heart. These mouse isoforms are highly homologous and contain the histidine motif. The physiological roles of these different enzyme isoforms are currently not understood.

Saccharomyces cerevisiae (yeast) contains a single, essential gene (OLE1) that codes for a desaturase enzyme that is homologous to mouse SCDs. A yeast mutant lacking the OLE1 gene is incapable of growing in the absence of unsaturated fatty acids (UFAs). Transformation with an exogenous gene containing desaturase activity would complement an OLE1 deficient mutant.

SCD has been identified as a possible drug target for the treatment of several diseases, including obesity. The development of efficient drugs that can regulate the activity of this enzyme can only be accomplished upon understanding of its function. Currently, little is known about the many factors that induce or inhibit SCD expression. The characterization of the different SCD isoforms has been impaired by the lack of a system that allows the isolation of each enzyme. The state of the art involves isolation of SCD using impure microsomal preparations obtained from liver homogenates.

It would be advantageous to develop a system that will enable the determination of the SCD enzymatic activity in vitro. This knowledge could lead to a better understanding of the physiological role of SCD and its isoforms in vivo. The generation of such model expression system could also be used for identification of the roles of the proteins involved in a multi-protein enzyme complex such as desaturase. The present invention addresses these and other related needs.

### SUMMARY OF THE INVENTION

The present invention provides vectors for expression of enzymes involved in desaturation of fatty acids. The vectors may include one or more genes encoding one or more components of a desaturase complex. In one embodiment, the vectors may include equal proportions of each of a stearoyl-CoA desaturase (SCD) coding sequence, cytochrome b5 coding sequence, and cytochrome b5 reductase coding sequence.

The present invention also relates to an expression system that expresses one or more enzymes involved in desaturation of fatty acids. The expressed enzymes may form a significant part of the protein complex required for desaturation of fatty acids. In one aspect, various isoforms of the enzymes are expressed in order to determine the functions of those isoforms. In one preferred aspect, the expression system is heterologous (i.e. the expressed enzymes are of heterologous origin).

In a different aspect of the invention, different variants of the enzymes are expressed, to determine their respective functions. When variant forms of enzymes are expressed, the activity of these forms of enzymes may be investigated. For example, the variants may include isoforms, homologs, mutated enzymes, truncated enzymes, etc.

When more than one enzyme is expressed to form a desaturase expression system, by using different forms of enzymes, the activity of each individual enzyme that is a part of the desaturase complex can be determined. In other words, expression of multiple components of the desaturase complex enables the studying of the activity of distinct forms of those components (native enzymes, isoforms, mutants, truncated enzymes, etc.).

### BRIEF DESCRIPTION OF THE DRAWINGS

FIG. 1 depicts a restriction enzyme map of an exemplary expression vector pHSCD5 for expression of human stearoyl-CoA desaturase (SCDh5) in yeast vector.

FIG. 2 depicts a restriction enzyme map and a sequence of an exemplary expression vector for expression of soluble human cytochrome b5 in E. coli.

FIG. 3 shows optical spectra of purified human cytochrome b5 reductase and human cytochrome b5 (cyt b5) from pPSb5r in Escherichia coli.

FIG. 4 shows a restriction enzyme map of an exemplary expression vector for expression of human cytochrome b5 in yeast.

FIG. 5 shows a restriction enzyme map of an exemplary expression vector for expression of human cytochrome b5 reductase in E. coli.

FIG. 6 shows a restriction enzyme map of an exemplary expression vector for expression of human cyt b5 reductase in yeast.

FIG. 7 shows a restriction enzyme map of an exemplary expression vector for co-expression of SCDh5 and human cyt b5 in yeast.

FIG. 8 shows a restriction enzyme map of-an exemplary expression vector for co-expression of SCDh5 and human cyt b5 reductase in yeast.

FIG. 9 shows restriction enzyme maps of two exemplary variations of vectors for co-expression of SCDh5, human cyt b5, and human cyt b5 reductase in yeast.

FIG. 10 shows a restriction enzyme map depicting the placement of the GAL1 promoter for expression of SCDh5, LEU2 promoter for expression of human cyt b5, and TRP promoter for expression of human cyt b5 reductase.

FIG. 11 depicts a graph demonstrating the functional expression of SCDh1 and SCDh5 in yeast from the vector shown in FIG. 1.

FIG. 12 shows a restriction enzyme map of an exemplary expression vector for expression of mycobacterial DesA3 in wheat germ cell-free translation.

FIG. 13 shows a restriction enzyme map of an exemplary expression vector for expression of mycobacterial Rv3230c in wheat germ cell-free translation.

FIG. 14 is an image of an SDS-PAGE gel showing expression of DesA3 and Rv3230c in cell free translation from vectors shown in FIG. 12 and FIG. 13.

FIG. 15 shows a restriction enzyme map of an exemplary expression vector for expression of SCDh1 in wheat germ cell-free translation.

FIG. 16 shows a restriction enzyme map of an exemplary expression vector for expression of SCDh5 in wheat germ cell-free translation.

FIG. 17 is an image of an SDS-PAGE gel showing expression of human, mouse and mycobacterium SCD in cell free translation from vectors shown in FIG. 12, FIG. 15, and FIG. 16.

FIG. 18 shows a restriction enzyme map of an exemplary expression vector for expression of human cyt b5 in wheat germ cell-free translation.

FIG. 19 shows a restriction enzyme map of an exemplary expression vector for expression of human cyt b5 reductase in wheat germ cell-free translation.

FIG. 20 depicts a graph of band intensity (level of expression) indicating the time course for expression of native (full-length) mouse SCD1 () and a truncated mouse SCD1 (◯).

FIG. 21 is a schematic diagram of the operon structure of Mycobacterium tuberculosis near to the DesA3 and Rv3230c genes.

FIG. 22 shows a restriction enzyme map of an exemplary expression vector for constitutive expression of DesA3 in Mycobacterium smegmatis.

FIG. 23 shows a restriction enzyme map of an exemplary vector for inducible expression of Rv3230c in Escherichia coli.

FIG. 24 shows restriction enzyme maps of exemplary co-expression vectors for inducible expression of DesA3 and Rv3230c in Mycobacterium.

FIG. 25 shows an image obtained from a Packard Instant Imager (Packard, Meriden, Conn.) (top) for phosphorescence detection of radioactive decay and quantitative analysis (bottom) of duplicate trials for the conversion of [14C]-18:0-CoA to [14C]-18:1-CoA by recombinant mouse SCD1 in the presence of various combinations of recombinant preparations of cytochrome b5 and cytochrome b5 reductase, demonstrating stimulation of in vitro SCDm1 activity by addition of soluble domain of human cyt b5 and human cyt b5 reductase.

FIG. 26 shows images obtained from a Packard Instant Imager (Packard, Meriden, Conn.) for phosphorescence detection of radioactive decay and quantitative analysis (bottom) of duplicate trials for the conversion of [¹⁴C]-18:0-CoA to [¹⁴C]-18:1-CoA after expression of DesA3 from vector DesA3HispVV16 in Mycobacterium smegmatis, demonstrating stimulation of DesA3 activity by combination with Rv3230c.

### DETAILED DESCRIPTION OF THE INVENTION

General Overview

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as in Sambrook et al., 2000, Molecular Cloning: A Laboratory Manual, third edition, Cold Spring Harbor Laboratory Press, Cold Spring Harbor, N.Y.; Sambrook et al., 1989, Molecular Cloning: A Laboratory Manual, second edition, Cold Spring Harbor Laboratory Press, Cold Spring Harbor, N.Y.; Ausubel et al., 1987-2004, Current Protocols in Molecular Biology, Volumes 1-4, John Wiley & Sons, Inc., New York, N.Y.; Kriegler, 1990, Gene Transfer and Expression: A Laboratory Manual, Stockton Press, New York, N.Y.; Dieffenbach et al., 1995, PCR Primer: A Laboratory Manual, Cold Spring Harbor Laboratory Press, Cold Spring Harbor, N.Y., each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well known and commonly employed in the art. Standard techniques are used for cloning, DNA, RNA, and protein isolation, nucleic acid amplification, and nucleic acid and protein purification. Generally enzymatic reactions involving DNA ligase, DNA polymerase, restriction endonucleases and the like are performed according to the manufacturer's specifications.

Definitions

“Desaturases” refer to enzymes that remove two hydrogen atoms from adjacent carbons in an organic compound, creating a carbon/carbon double bond. Such enzymes can be found in humans and other eukaryotes (such as monkeys, rats, mice, zebrafish, cows, pigs, sheep, chickens, yeast, and others), in beneficial microorganisms (such as Streptomyces colieocolor, Streptomyces avermitilis and other bacteria that are responsible for the synthesis of a wide array of antibiotics), and in pathogens (such as Mycobacterium tuberculosis, Mycobacterium leprae, Mycobacterium bovis, Mycobacterium avis, and many other Gram-positive actinomycetes).

“Nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

“Nucleic acid sequence encoding” refers to a nucleic acid that directs the expression of a specific protein or peptide. The nucleic acid sequences include both the DNA strand sequence that is transcribed into RNA, and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences that may be introduced to provide codon preference in a specific host cell.

“Coding sequence” or “coding region” refers to a nucleic acid molecule having sequence information necessary to produce a gene product, when the sequence is expressed.

“Nucleic acid construct” or “DNA construct” refers to a coding sequence or sequences operably linked to appropriate regulatory sequences so as to enable expression of the coding sequence.

“Isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. One of skill will recognize that these values can be appropriately adjusted to determine corresponding identity of proteins encoded by two nucleotide sequences by taking into account codon degeneracy, amino acid similarity, reading frame positioning and the like.

“Substantial identity” of amino acid sequences for purposes of this invention normally means polypeptide sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 98.7%, or 99%. Polypeptides that are “substantially identical” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

A protein “isoform” is a version of a protein with some small differences. For example, the small differences may be a result of a splice variant of the protein, or they may be the result of some post-translational modification. Often, an isoform of an enzyme may have different catalytic properties than the native form of the enzyme.

A “desaturase system” or “desaturase complex” refers to two or more components that are mixed together in order to facilitate the desaturation of fatty acids. Such components may include enzymes, e.g. desaturase, cytochrome b5, cytochrome b5 reductase, and others. The components may further include other proteinaceous or non-proteinaceous molecules that are involved in desaturation of fatty acids. These components may interface together. The components of the desaturase system may be structurally or functionally related.

An “enzyme assay” or “enzymatic assay” refers to a standard laboratory method for measuring enzymatic activity. An enzymatic assay for determination of desaturase activity (“desaturation assay”) refers to a laboratory method for determining the activity of the enzyme involved in fatty acid desaturation (desaturase).

For example, the change in 18:1-CoA production can be used for determination of the desaturase activity. A method for assay of 18:1-CoA production is provided as follows. Radioactive fatty acyl-CoAs were obtained from American Radiolabeled Chemicals (St. Louis, Mo.). The reaction mixture contained 20 mM of potassium phosphate and 150-250 mM of NaCl in a total reaction volume of 200 μL. Aliquots (20 μL) of the M. smegmatis pVV16 or pVV6-DesA3 total lysate, supernatant or pellet fractions were added in various combinations with aliquots (15 μL) of supernatant fraction prepared from either E. coli pQE80 or pQE80-Rv3230c. The reaction was initiated by addition of 0.4 μmol of NADPH, 6 nmol of stearoyl-CoA, 0.03 μCi of [1-¹⁴C]-stearoyl-CoA and 0.2 nmol of FAD in a combined volume of 200 μL. The reaction was incubated at 37° C. for 1 h and stopped by the addition of 200 μL of 2.5 M KOH in ethanol. The mixture was heated at 80° C. for 1 h and acidified by the addition of 280 μL of formic acid. The saponified fatty acids were extracted with 700 μL of hexane, 200 μL of the extract was evaporated to dryness, resuspended in 50 μL of hexane and separated into saturated and unsaturated acids on a 10% AgNO₃-impregnated thin-layer chromatography plate using chloroform:methanol:acetic acid:water (90:8:1:0.8) as the developing solvent. Radioactivity was counted by phosphorimaging using a Packard Instant Imager (Packard, Meriden, Conn.) for 30-60 min. Samples prepared in this manner gave ˜200 total imager units for the major radioactive bands detected, which is within the linear response range of the instrument. Reactions performed with stearoyl-CoA were also treated by thin-layer chromatography as described above, and the individual bands were extracted from the plate, methylated and analyzed by GC/MS to determine fatty acid content.

Desaturases

The present invention provides methods for expression of various desaturases as part of the desaturase expression system. The desaturases may be eukaryotic (e.g. of human, animal, or plant origin) and prokaryotic (e.g. of bacterial origin). For example, desaturases useful for practicing of the invention include soluble desaturases and membrane-bound desaturases, acyl-lipid desaturases, acyl-coenzyme A (acyl-CoA) desaturases, acyl-acyl carrier protein (ACP) desaturases, and other desaturases. Preferably, the desaturase is a stearoyl-CoA desaturase.

Oxidoreductases

The present invention provides methods for expression of various oxidoreductases as part of the desaturase expression system. Oxidoreductases are enzymes of EC class 1. Oxidoreductases catalyze oxidation-reduction reactions, which entail the transfer of electrons from a substrate that becomes oxidized (electron donor) to a substrate that becomes reduced (electron acceptor).

The oxidoreductases can be substrate-specific, which means that they can preferably catalyze the oxidation-reduction reactions of specific substrates. For example, an oxidoreductase that is specific for a CH—CH group of donors preferably catalyzes oxidation-reduction reactions of substrates containing CH—CH groups. Preferably, the oxidoreductases for practicing the invention are cytochrome b5, cytochrome b5-like proteins, and cytochrome b5 reductase, Mycobacterium tuberculosis H37Rv oxidoreductase Rv3230c and related proteins from other pathogenic prokaryotes.

Vectors

The invention involves genetically engineering a system for the expression of enzymes involved in fatty acid desaturation. The genetic engineering may include increasing the amount of enzymes involved in desaturation. However, in other instances, the genetic engineering may additionally include expression of other non-enzymatic components that are involved in desaturation.

Engineering of the desaturase expression system involves providing for the expression of one or more heterologous genes that encode protein(s) involved in desaturation. The heterologous gene may be a gene that is not naturally present in the desaturase system, or it may be a gene that is naturally present but is placed in a different genetic context (e.g., the coding region of the gene is operably linked to a promoter that is not the gene's natural promoter). Typically, the heterologous gene or the resulting protein will have one or more properties differing from the gene in its natural genetic environment.

One method of expression of proteins of the desaturase system of this invention is through the use of vectors such as plasmids, phage, phagemids, viruses, artificial chromosomes and the like. Preferred vectors are expression vectors. Expression vectors contain a promoter that may be operably linked to a coding region. A gene or coding region is operably linked to a promoter when transcription of the gene initiates from the promoter. More than one gene may be operably linked to a single promoter. In preferred embodiments, at least one desaturase gene and at least one oxidoreductase gene are both operably linked to the same promoter. In other preferred embodiments, at least one desaturase gene, at least one cytochrome b5 gene, and at least one cytochrome b5 reductase gene are operably linked to the same promoter. In other preferred embodiments, each of the genes is operably linked to a different promoter. In one aspect, the vector is introduced into an organism that is suitable for expression of the desaturase system.

A variety of expression vectors may be used for expression in E. coli, insect, yeast, or mammalian cells. Expression vectors that may be used include, but are not limited to, Gateway® Destination vectors (Invitrogen, Carlsbad, Calif.), pQE-30, pQE-40, and pQE-80 series (Qiagen, Valencia, Calif.), pUC19 (Yanisch-Perron et al., 1985, Gene 33: 103-119), pBluescript 11 SK+ (Stratagene, La Jolla, Calif.), the pET system (Novagen, Madison, Wis.), pLDR20 (ATCC 87205), pBTrp2, pBTac1, pBTac2 (Boehringer Ingelheim Co., Ingelheim, Germany), pLSA1 (Miyaji et al., 1989, Agric. Biol. Chem. 53: 277-279), pGEL1 (Sekine et al., 1985, Proc. Natl. Acad. Sci. USA. 82: 4306-4310), and pSTV28 (manufactured by Takara Shuzo Co., Shimogyo-ku, Kyoto 600-8688, Japan). When a yeast strain is used as the host, examples of expression vectors that may be used include pYEST-DES52 (Invitrogen), YEp13 (ATCC 37115), YEp24 (ATCC 37051), and YCp50 (ATCC 37419). When insect cells are used as the expression host, examples of expression vectors that may be used include pFASTBac1 (Invitrogen, Carlsbad, Calif.), pVL1393 (BD Biosciences, Franklin Lakes, N.J.) and pIEX (Novagen, Madison, Wis.).

Alternatively, expression kits might be utilized for cell-free protein expression. For example, the EasyXpress Protein Synthesis Mini Kit, the EasyXpress Protein Synthesis Mega Kit (Qiagen), the In vitro Director™ System (Sigma-Aldrich, St. Louis, Mo.), the TnT Sp6 High-Yield Protein Expression System (Promega; Madison, Wis.) or the WePro lysate (Cell Free Sciences, Yokohama, Japan) might be used. Examples of expression vectors used for cell-free protein expression include pIX4 (Qiagen; Valencia, Calif.) and pEU (Cell Free Sciences, Yokohama, Japan).

In one example, cell-free expression of one or more components of the desaturase system obviates the need for assembly of multiple genes in an expression vector to achieve co-expression. Instead, transcribed mRNA from plasmid can be added to achieve any ratio of translated protein. That is why in some examples it may not be necessary to put multiple genes into expression plasmid backbones.

Expression of the components of the desaturase system is controlled with the use of desirable promoters. Essentially any promoter may be used as long as it can be expressed in the engineered organism. A preferred promoter for E. coli is the lambda PR promoter. In the presence of the product of the lambda C_{I} repressor gene, transcription from the lambda PR promoter may be controlled. At temperatures below 37° C., the repressor is bound to the lambda PR promoter and transcription does not occur. At temperatures above 37° C. the repressor is released from the lambda PR promoter and transcription initiates. Thus, by growing the organism containing the vector at 37° C. or above, the genes are expressed.

A preferred promoter for E. coli is the lac promoter. In the presence of allolactose, an alternative product of the metabolism of lactose by beta-galactosidase, transcription from the lac promoter may be controlled. In the absence of allolactose, the lac repressor is bound to the lac operator and transcription does not occur. In the presence of allolactose, the repressor is released from the lac operator and transcription initiates. Thus, by growing the organism containing the vector containing lac operator sequences and lac repressor in the presence of allolactose, the genes are expressed.

When the organism is a yeast cell, any promoter expressed in the yeast strain host can be used. Examples include the gal 1 promoter (GAL1), leu2 promoter (LEU2), tryptophan promoter (TRP), gal 10 promoter, heat shock protein promoter, MF alpha 1 promoter, and CUP 1 promoter.

A ribosome-binding sequence (RBS) (prokaryotes) or an internal ribosome entry site (IRES) (eukaryotes) may be operably linked to the gene. The RBS or IRES is operably linked to the gene when it directs proper translation of the protein encoded by the gene. It is preferred that the RBS or IRES is positioned for optimal translation of the linked coding region (for example, 6 to 18 bases from the initiation codon). In vectors containing more than one gene, it is preferred that each coding region is operably linked to an RBS or IRES. A preferred RBS is AGAAGGAG.

The gene or genes encoding components of the desaturase complex may also be operably linked to a transcription terminator sequence. A preferred terminator sequence is the T7 terminator (pET15b; Novagen, Madison, Wis.).

The coding region of the gene may be altered prior to insertion into or within the expression vector. These mutants may include deletions, additions, and/or substitutions. When alterations are made, it is preferred that the alteration maintains the desired enzymatic function or specificity of the enzyme. However, in certain embodiments, it may be desired to alter the specificity of the enzyme. For example, one may wish to alter the desaturase such that the activity of the enzyme is changed.

When a heterologous gene is to be introduced into an organism that does not naturally encode the gene, optimal expression of the gene may require alteration of the codons to better match the codon usage of the host organism. The codon usage of different organisms is well known in the art.

The coding region also may be altered to ease the purification or immobilization. An example of such an alteration is the addition of a “tag” to the protein. Examples of tags include FLAG, polyhistidine, biotin, T7, S-protein, myc-, and GST (Novagen; pET system). In one preferred embodiment, the gene is altered to contain a hexo-histidine tag in the N-terminus. The protein may be purified by passing the protein-containing solution through a Ni²⁺ column.

In other embodiments, the coding regions of two or more enzymes are linked to create a fusion protein. In preferred embodiments, a desaturase-cytochrome b5 fusion protein is encoded. In another preferred embodiment, the fusion protein comprises a desaturase-cytochrome b5 reductase.

In further preferred embodiments, the expression vector of the present invention comprises at least one polynucleotide sequence encoding a desaturase and at least one polynucleotide sequence encoding an oxidoreductase. The plasmid may also encode one or more enzymes that facilitate fatty acid desaturation.

Expression of a Desaturase System

The optimal function of a multi-protein enzyme complex such as desaturase requires the presence of all members of the protein complex. In one aspect, the invention provides a recombinant expression system that includes components of a desaturation complex that are involved in fatty acid desaturation. Preferably, these components are enzymes. At minimum, the desaturation complex includes two enzymes: desaturase and oxidoreductase. In various aspects of the invention, different forms of desaturases and oxidoreductases may be used. For example, they may be native (full-length), truncated, mutated, or otherwise modified by methods known in the art. With respect to the expression system, the desaturase and oxidoreductase may be homologous, heterologous, or may constitute mixtures thereof (i.e. one or more enzymes are homologous, whereas other one or more enzyme are heterologous).

In one aspect, the present invention provides a multi-protein desaturase complex from Mycobacterium tuberculosis. The complex includes Rv3229c and Rv3230c gene products. The complex may also include additional gene products. In one embodiment, individual plasmids may express individual gene products that form the desaturase complex. For example, if several plasmids are used for expression, each plasmid may carry one or more genes encoding one or more components of a mycobacterial desaturase complex. Alternatively, a vector may carry more than one gene encoding more than one component of a mycobacterial desaturase complex. Thus, expression of multiple proteins of a mycobacterial desaturase complex may be achieved with the use of only one vector.

In a different aspect, the invention provides multiple vectors that express components of a mammalian desaturase complex. In one embodiment, individual plasmids may express individual gene products that form the desaturase complex. For example, if several plasmids are used for expression, each plasmid may carry one gene encoding one or more components of a desaturase complex. Alternatively, one vector may carry more than one gene, each gene encoding one or more components of a mammalian desaturase complex. Thus, expression of multiple proteins of a desaturase complex may be achieved with the use of only one vector.

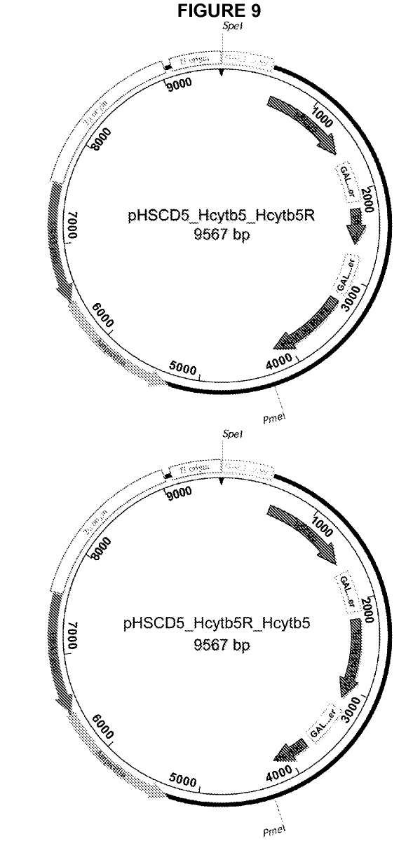

In one preferred embodiment, the genes encoding components of a desaturase complex are present in equal proportions. This can be accomplished in a variety of ways, for example by using one vector that has equal proportions of genes from a desaturase complex. These genes may be inserted under the control of same control elements for gene expression. One vector may carry sequences encoding more than one component of the complex. In one embodiment, the vector might carry three components of the desaturase complex, present in a certain ratio. In a preferred embodiment, the ratio of SCD, cytochrome b5, and cytochrome b5 reductase coding sequences is 1:1:1, as shown in FIG. 9. Alternatively, individual genes of the desaturase complex may be expressed using separate plasmids. In that case, each plasmid of the expression system may carry at least one gene encoding a component of the complex, while all plasmids have identical control elements for gene expression.

In a different embodiment, the invention provides a cell-free expression system for desaturases, where genes that encode the desaturase complex are added to the system, proteins are expressed, and enzymatic activities are determined. The proteins that form the desaturase complex may be introduced by any method known in the art. Preferably, proteins that form the desaturase complex include SCD, cytochrome b5, and cytochrome b5 reductase or Rv3229c (mycobacterial DesA3) and Rv3230c (mycobacterial DesA3 oxidoreductase).

The desaturase may exist as a separate enzyme or may be a genetic fusion with an oxidoreductase domain.

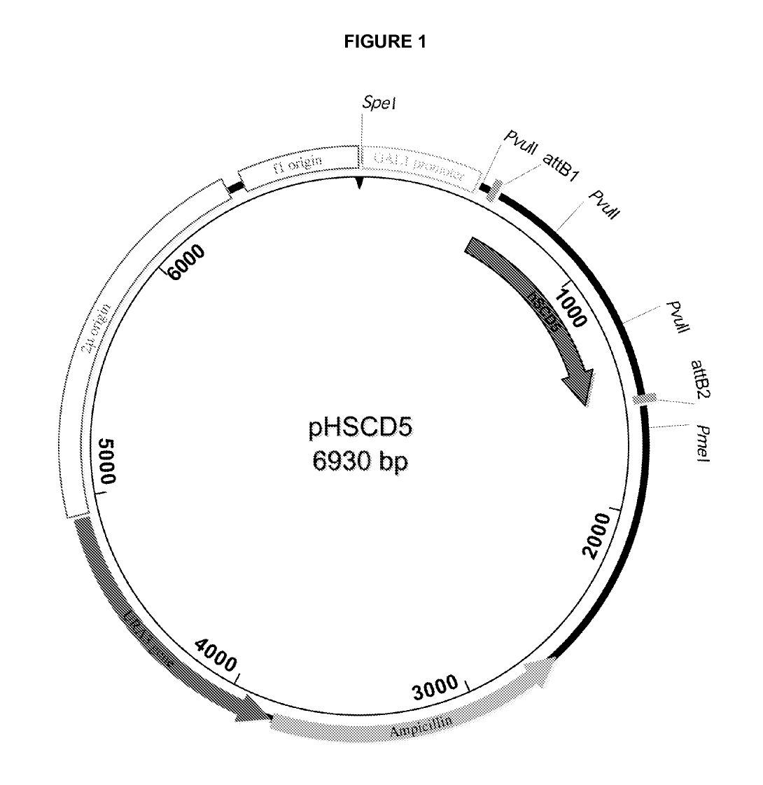

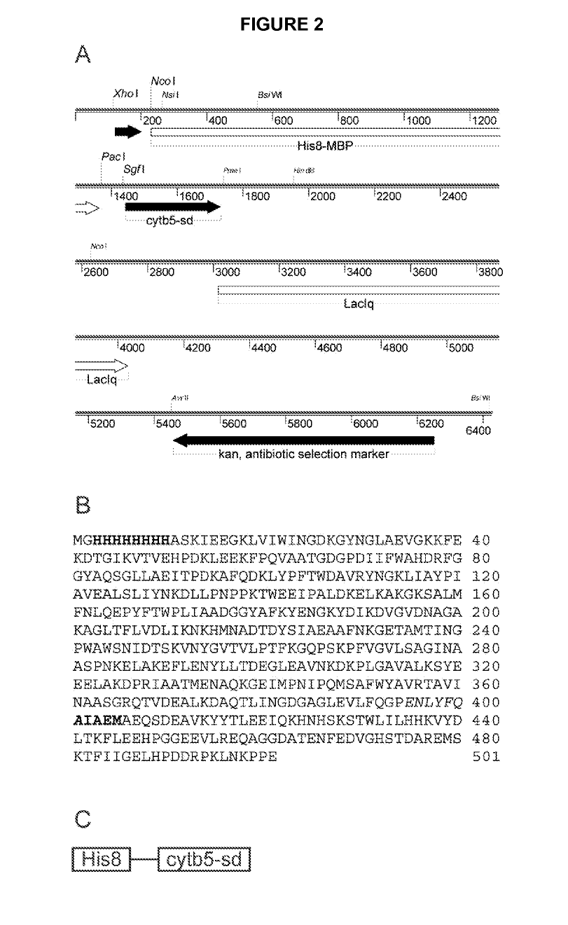

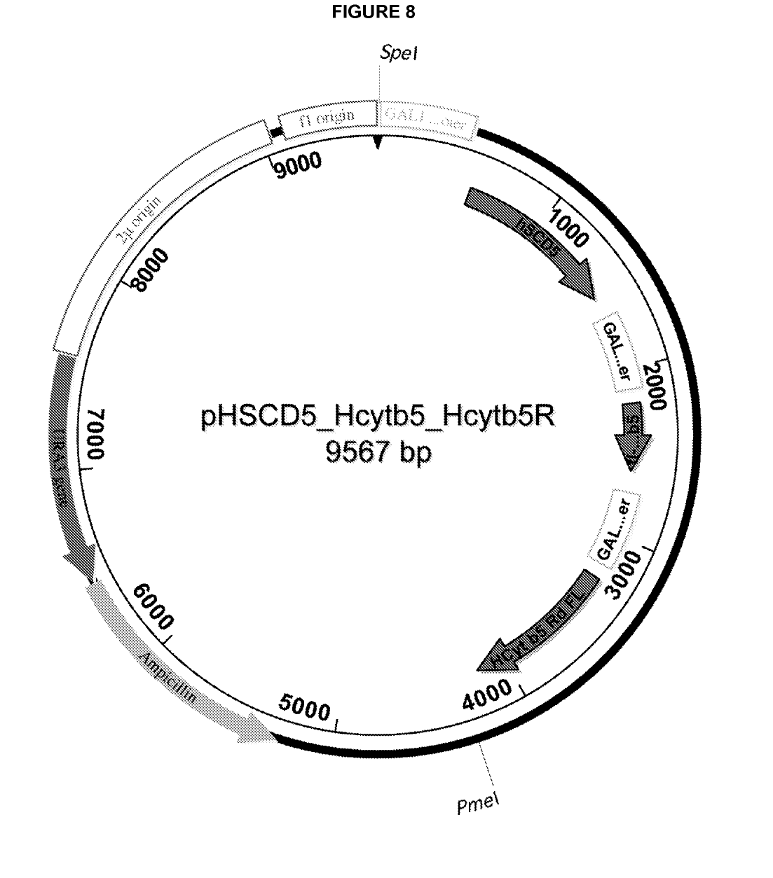

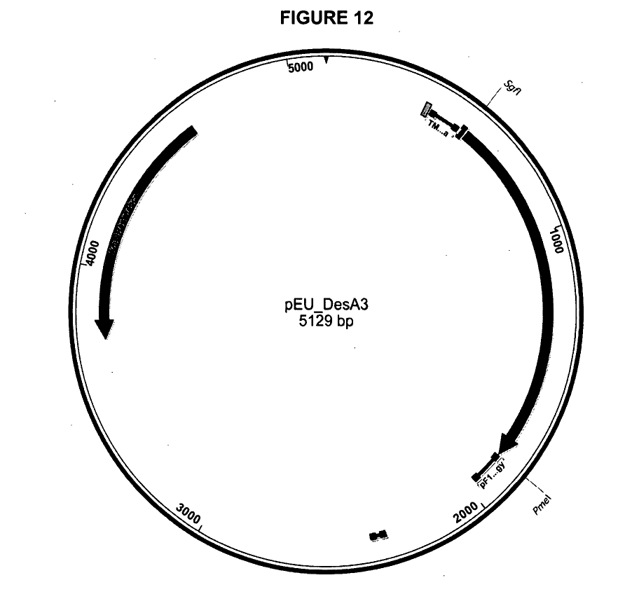

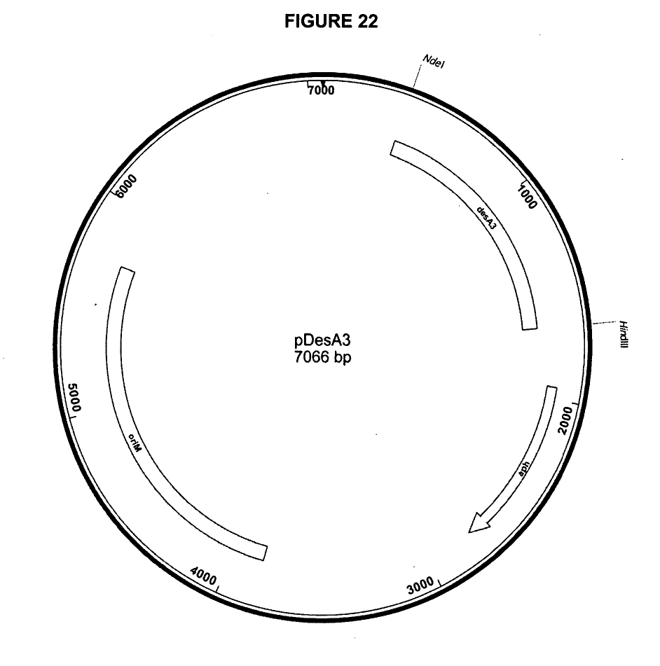

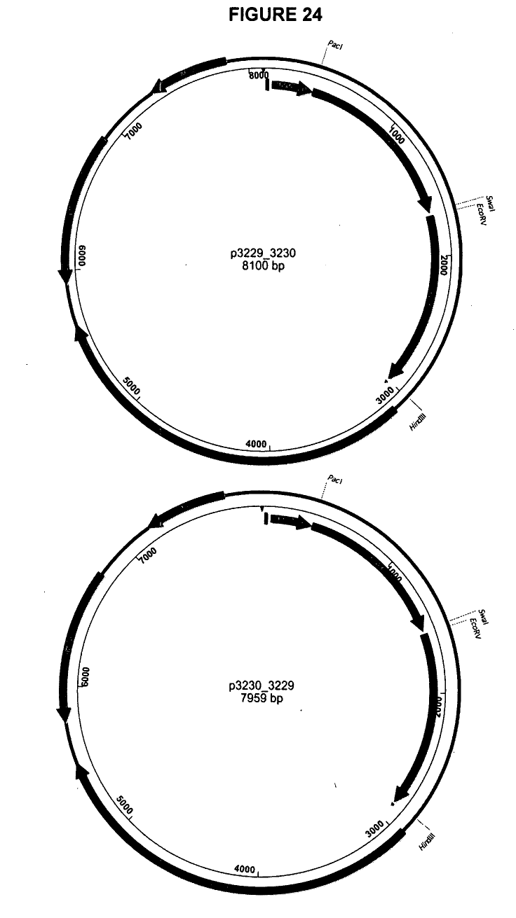

Some examples of expression vectors useful for practicing the present invention are shown below. For example, the plasmids shown in FIGS. 1, 4, 6, 7, 8, 9, and 10 for expression in yeast derive from Invitrogen Gateway vectors pDONR221 and pYES-DEST52. The plasmids shown in FIGS. 2, 5, and 23 for expression in Escherichia coli derive from QIAGEN vector pQE80. They can be modified as described by Blommel et al., 2007, Biotechnol. Prog. 23: 585-598; Blommel and Fox, 2007, Protein Expr. Purif. 55: 53-68; Blommel et al., 2006, Protein Expr. Purif. 47: 562-570. The plasmids shown in FIGS. 12, 13, 15, 16, and 18 derive from Cell Free Sciences (75-1, Ono-cho, Leading Venture Plaza Tsurumi-ku, Yokohama, 230-0046) vector pEU-His as modified by Blommel et al., 2006, Protein Expr. Purif 47: 562-570. The vector in FIG. 22 derives from vector pVV16 (Phetsuksiri et al., 2003, J. Biol. Chem. 278: 53123-53130). The co-expression vector in FIG. 24 derives from vector pSE100 described in Ehrt et al., 2005, Nucleic Acids Res. 33: e21. The co-expression vector in FIG. 24 provides PacI, SwaI, EcoRV, and HindIII sites compatible with cloning the genes Rv3229c (DesA3) and Rv3230c (reductase) under control of the UV15TetO promoter.

Genes are cloned by standard PCR methods to incorporate restriction sites before the start and stop codons. For example, the co-expression vector p3229_{—}3230 provides the Rv3229c gene cloned adjacent to the promoter by PacI and SwaI restriction cloning and the Rv3230c gene cloned distal to the promoter by EcoRV and HindIII. The co-expression vector p3230_{—}3229 provides the Rv3230c gene cloned adjacent to the promoter by PacI and SwaI restriction cloning and the Rv3230c gene cloned distal to the promoter by EcoRV and HindIII restriction cloning. FIG. 24 shows two different arrangements of the gene relative to the promoter.

The invention also provides an assay system for determination of desaturase activity. Preferably, the system includes at least one expression vector, preferably a vector that includes genes encoding SCD and an oxidoreductase. In a preferred embodiment, the vector includes sequences encoding SCD, cytochrome b5, and cytochrome b5 reductase, with the ratio of coding sequences being 1:1:1. Desaturase activity assays may be conducted with variants of the components of the expression system. For example, different expression vectors for expression of one or more of the components may be used. Also, the individual components that are expressed may be varied. For example, in another preferred embodiment, Rv3229c (mycobacterial DesA3) and Rv3230c (mycobacterial DesA3 oxidoreductase) are combined in a ratio of 10:1. As another example, a homolog or a mutant protein may be expressed as a part of the desaturase complex, to study its role in the enzymatic reactions.

In some embodiments, different variants of the genes or proteins are introduced. These include homologs, mutants, proteins with amino acids substitutions, etc., depending on the objective of the investigation.

It is further possible to optimize the cell-free expression system of this invention by stabilizing each of the components of the desaturase system in its own stabilizing buffer, using methods known in the art.

In a further aspect the present invention includes a desaturase complex together with at least one co-factor, isolated in its pure form, and then added to the desaturase complex.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

It is to be understood that this invention is not limited to the particular methodology, protocols, patients, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

### Example 1

Expression System for Human Stearoyl-CoA Desaturase

A plasmid containing the gene for human stearoyl-CoA desaturase (hSCD1) is obtained from the Mammalian Gene Collection. PCR reactions are used to clone hSCD1 and to add the codons for specific amino acid sequences that provide optimal expression in yeast. First, the codons for a 27-amino acid sequence corresponding to the native yeast desaturase endoplasmic reticulum localization sequence are added. Then, a ribosomal binding site and the recombination sites required for Gateway® (Invitrogen) cloning are introduced. The modified hSCD1 gene is initially transferred into the pDONR221 entry plasmid, and then transferred into the commercial yeast expression plasmid pYES-DEST52 following the standard Gateway® cloning procedure. Note that one skilled in the art will know to use expression systems other than Gateway® to achieve the goal of expressing the proteins that form the desaturase complex. The hSDC1 protein may also be expressed without the 27-amino acid sequence in yeast. Human stearoyl-CoA desaturase isoform 5 (SCDh5) can be cloned in the same manner (FIG. 1).

The plasmid containing the SCDh5 gene contains unique SpeI and PmeI sites that can be used to add additional genes to the expression plasmid. These genes may include other desaturases or oxidoreductases such as cytochrome b5 or cytochrome b5 reductases.

Expression System for Mouse Stearoyl-CoA Desaturase Isoforms

The genes for the four SCD isoforms from mice are isolated as cDNAs from mouse liver (mSCD1), brain (mSCD2), Harderian gland (mSCD3), and heart (mSCD4). PCR reactions are used to clone each mouse SCD gene and to add codons for specific amino acid sequences to provide optimal expression in yeast. First, the codons for a 27-amino acid sequence corresponding to the native yeast desaturase endoplasmic reticulum localization sequence are added. Then, a ribosomal binding site and the recombination sites required for Gateway® cloning are introduced.

The modified mouse SCD genes are initially transferred into the pDONR221 entry plasmid, and then transferred into the commercial yeast expression plasmid pYES-DEST52 following the standard Gateway® cloning procedure, to create plasmids pSCDm1, pSCDm2, pSCDm3, and pSCDm4 (FIG. 1). FIG. 1 depicts expression vector pSCDh5 for expression of human stearoyl-CoA desaturase. Alternatively, this vector can be engineered to contain SCDh1, SCDm1, SCDm2, SCDm3, or SCDm4. Thus, the Gateway system can be used to transfer other desaturase genes into this plasmid, including mouse stearoyl-CoA desaturase isoforms to give other expression plasmids named pSCDm1, pSCDm2, pSCDm3 and pSCDm4.

Expression System for Soluble Human Cytochrome b5

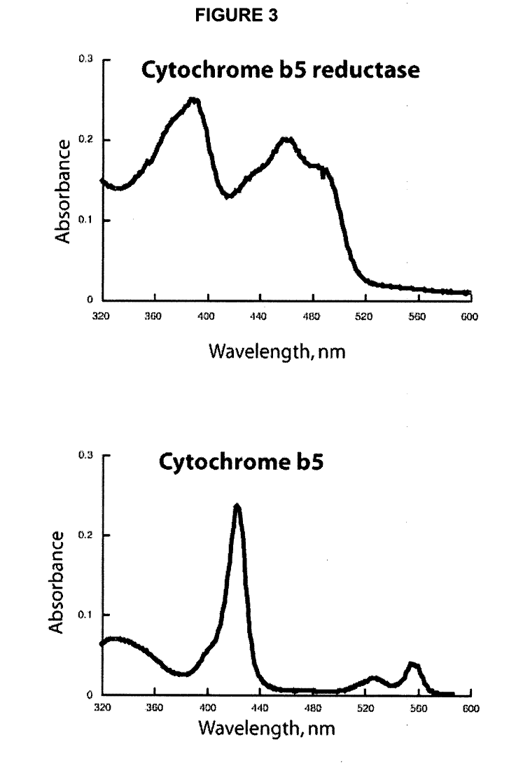

FIG. 2 shows a vector that can be used to express human cytochrome b5 in E. coli. The gene for human cytochrome b5 is purchased from the Mammalian Gene Collection. The gene is engineered by PCR such that the membrane interacting region at the C-terminus of the native enzyme is deleted in order to produce a soluble enzyme. The gene is also engineered to encode an N-terminal His8-tag and FlexiVector (Promega) restriction sites at the 5′ and 3′ ends. Other tags are possible. The engineered gene is transferred into a Flexi Vector plasmid for bacterial expression (FIG. 2). The plasmid is named pPSb5. The protein is expressed in Escherichia coli. The expressed fusion protein can be purified in high yield by Ni-affinity purification followed by gel filtration chromatography (FIG. 3).

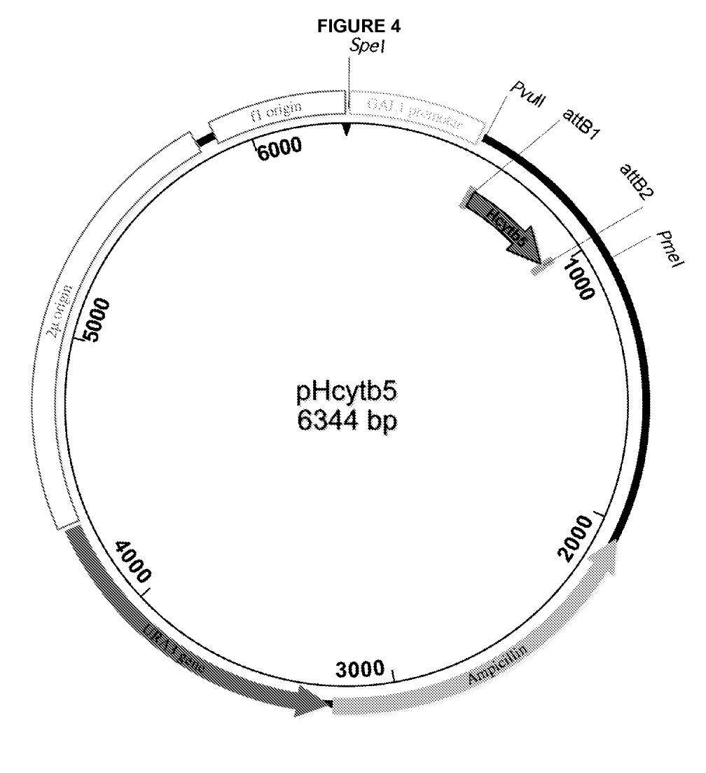

Alternatively, the gene for cytochrome b5 can initially be transferred into the pDONR221 entry plasmid, and then transferred into the commercial yeast expression plasmid pYES-DEST52 following the standard Gateway® cloning procedure. This vector is shown in FIG. 4. Note that one skilled in the art will know to use expression systems other than Gateway® to achieve the goal of expressing the proteins that form the desaturase complex.

Expression System for Soluble Human Cytochrome b5 Reductase

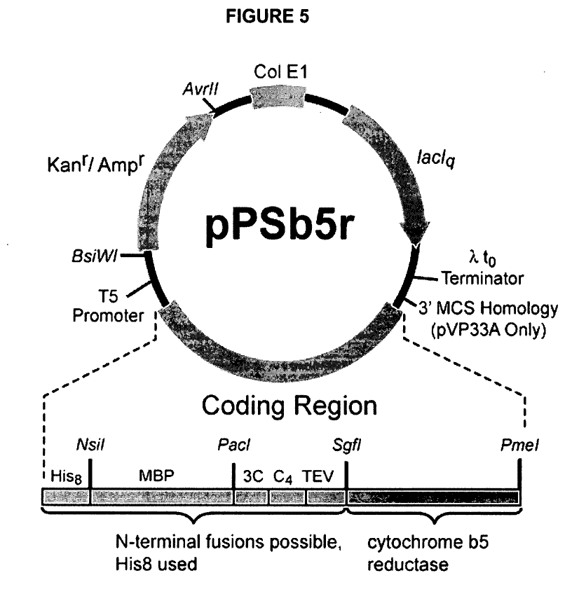

The gene for human cytochrome b5 reductase is purchased from the Mammalian Gene Collection. The gene is engineered by PCR such that the membrane interacting region at the N-terminus of the native enzyme is deleted in order to produce a soluble and active enzyme. The gene is also engineered to encode an N-terminal His8-tag and Flexi Vector restriction sites at the 5′ and 3′ ends. Other tags are possible. The engineered gene is transferred into a Flexi Vector plasmid for bacterial expression (FIG. 5). The plasmid is named pPSb5r. The protein is expressed in Escherichia coli. The expressed fusion protein is purified in high yield by Ni-affinity purification followed by gel filtration chromatography (FIG. 3).

Alternatively, the gene for cytochrome b5 reductase is subcloned into the pDONR221 entry plasmid, and then transferred into the commercial yeast expression plasmid pYES-DEST52 following the standard Gateway® cloning procedure. This vector is shown in FIG. 4. Note that one skilled in the art will know to use expression systems other than Gateway® to achieve the goal of expressing the proteins that form the desaturase complex.

Binary Expression of Human SCD1 and Human Cytochrome b5

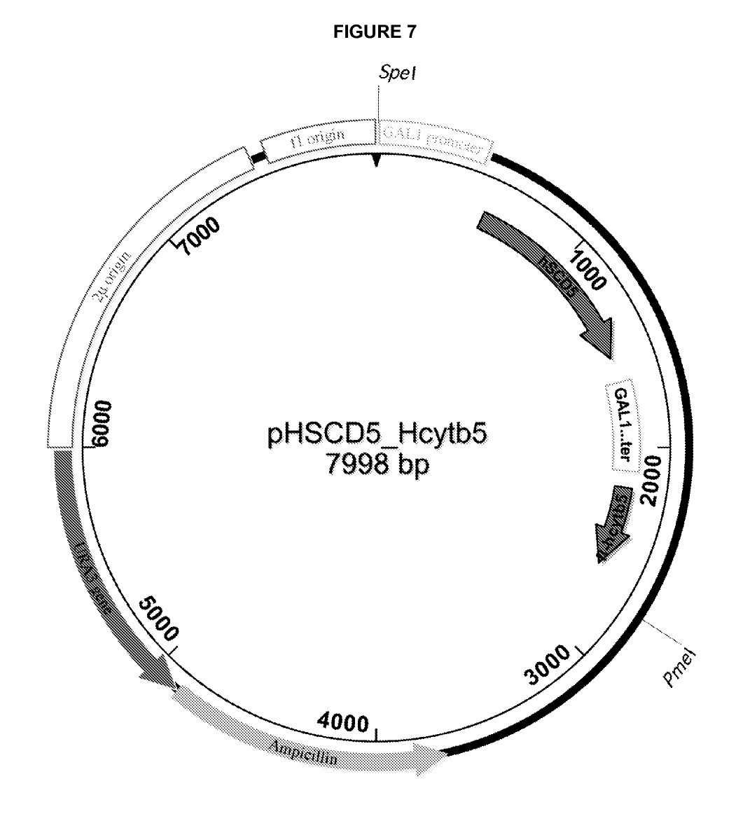

The plasmid pSCDb5 for binary expression of human SCDh5 and human cytochrome b5 (FIG. 7) is created by insertion of the SpeI and PmeI fragment of pHcytb5 (FIG. 4) into the Pmel-digested plasmid pHSCD5 (FIG. 1). The cohesive end yielded by Spel must be converted to a blunt end by endonuclease digestion. The human SCD1 gene can be replaced by any of the mouse isoform genes contained in pSCDm1, pSCDm2, pSCDm3 or pSCDm4 as desired to create heterologous binary expression vectors pSCDm1b5, pSCDm2b5, pSCDm3b5 or pSCDm4b5.

Binary Expression of Human SCD1 and Human Cytochrome b5 Reductase

The plasmid pSCDb5r for binary expression of human SCD1 h5 and human cytochrome b5 reductase is created by insertion the SpeI and PmeI fragment of pHcytb5 (FIG. 4) into the Pmel-digested plasmid pHSCD5 (FIG. 1). The human SCDh5 gene can be replaced by any of the mouse isoform genes contained in pSCDm1, pSCDm2, pSCDm3 or pSCDm4 as desired to create heterologous binary expression vectors pSCDm1b5r, pSCDm2b5r, pSCDm3b5r or pSCDm4b5r.

Construction of an Expression System for Complete Stearoyl-CoA Desaturase

The expression plasmid for the complete human stearoyl CoA desaturase system is created by ligation of the natural cytochrome b5 reductase gene into the PmeI site of pSCDb5. This plasmid is named pSCDcx (FIG. 9, top). An alternative orientation of the cytochrome b5 and cytochrome b5 reductase genes is obtained by ligation of the natural cytochrome b5 gene into the PmeI site of pSCDb5r. This plasmid is named pSCDxc (FIG. 9, bottom). The two vectors give two arrangements of the cytochrome b5 and cytochrome b5 reductase genes relative to the desaturase gene. The orientation of these genes is not specified as blunt cloning has been used.

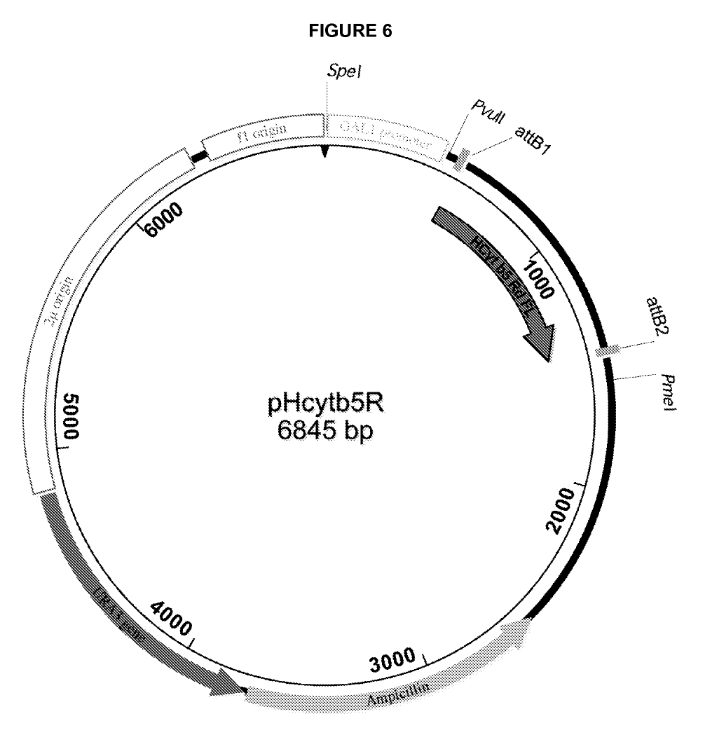

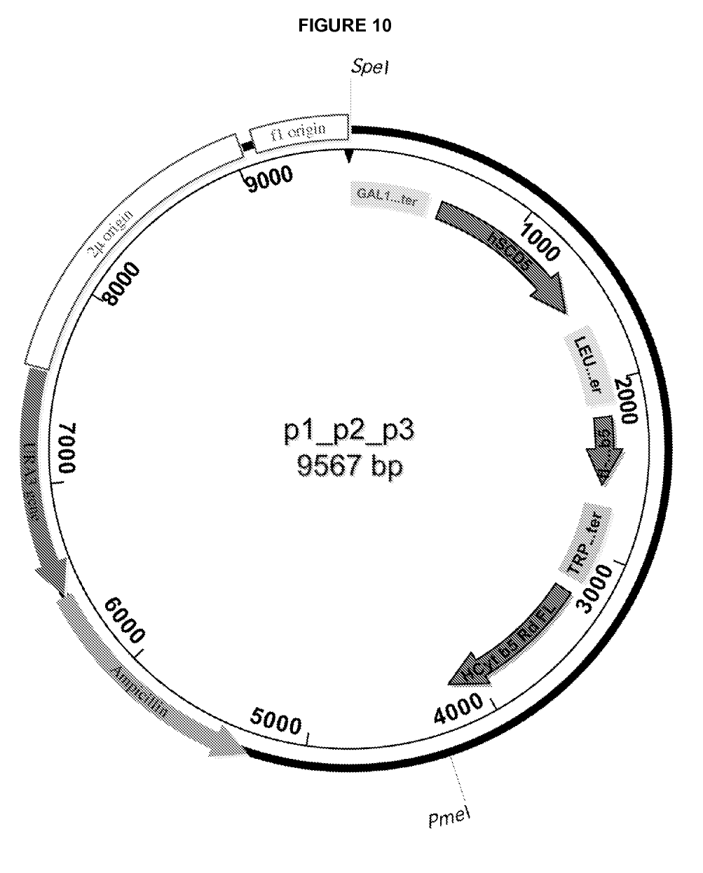

Alternative promoters can be added to the plasmids for individual expression of human cyt b5 and human cyt b5 reductase using the unique SpeI and PvuII sites (FIG. 4 and FIG. 6). Standard PCR amplification and restriction digestion methods are used to replace the GAL1 promoter with other yeast promoters. FIG. 10 provides an example of how this approach gives a coexpression vector for three genes with each having a different expression promoter.

### Example 2

Materials

Total RNA is obtained from mouse liver (SCD1), brain (SCD2), Harderian gland (SCD3), and heart (SCD4) using the TRIzol reagent (Invitrogen). AMV Reverse Transcriptase is from Promega (Madison, Wis.), and AccuPrime™ Pfx DNA polymerase is from Invitrogen (Carlsbad, Calif.).

TABLE 1

Custom designed primers utilized in the three-
step PCR for SCD cloning

| | | SEQ | |
|---|---|---|---|
| | | ID | |
| Name | Sequence | NO | Primer Info |
| Gene- | 5′-CCA AAG GAT GAC TCT | 5 | Forward primer |
| specific | GCC AGC AGT GGC ATT GTC | | containing |
| | GAC | | gene specific |
| | (+ gene specific | | overlap region |
| | region)-3′ | | plus portion |
| | | | of OLE1 start- |
| | | | er sequence. |
| 2^{nd} | 5′-CCA ACT TCT GGA ACT | 6 | Forward primer |
| Forward | ACT ATT GAA TTG ATT GAC | | containing re- |
| | GAC CAA TTT CCA AAG GAT | | mainder of |
| | GAC TCT GCC-3′ | | OLE1 starter |
| | | | sequence. |
| 3^{rd} | 5′-GGGG ACA AGT TTG TAC | 7 | Forward primer |
| Forward | AAA GCA GGC TCC AATA ATG | | containing ri- |
| | TCT CCA ACT TCT GGA ACT | | bosomal bind- |
| | ACT ATT G-3′ | | ing site and |
| | | | recombination |
| | | | site for |
| | | | Gateway ® |
| | | | cloning. |
| Reverse | 5′-GGGG AC CAC TTT GTA | 8 | Reverse primer |
| | CAA GAA AGC TGG GTC | | containing re- |
| | (+ gene specific region | | combination |
| | [lacking stop codon])-3′ | | site for |
| | | | Gateway ® |
| | | | cloning. |

The custom designed primers (Table 1) utilized in the three reactions are synthesized by and purchased from Integrated DNA Technologies (Coralville, Iowa). The gene-specific primer (SEQ ID NO:1) is used in the first PCR reaction to isolate the SCD gene and incorporate a portion of the OLE1 starter sequence. The 2^{nd} Forward primer (SEQ ID NO:2) is used in the second PCR reaction to incorporate the remainder of the OLE1 starter sequence. The 3^{rd} Forward primer (SEQ ID NO:3) is used in the third PCR reaction to incorporate a ribosomal binding site and the recombination site for Gateway® cloning at the 5′ end of the gene. The Reverse primer (SEQ ID NO:4) is used in all three PCR reactions to incorporate a recombination site for Gateway® cloning at the 3′ end of the gene. The stop codon is eliminated from the open reading frame using the Reverse primer such that the 6×His tag from the pYES-DEST52 plasmid would be incorporated.

Gene Cloning

Total RNA is obtained from mouse liver (SCD1), brain (SCD2), Harderian gland (SCD3), and heart (SCD4) using the TRIzol reagent. AMV Reverse Transcriptase is used to generate cDNA from the total RNA. Each plasmid is then used as the template in a three-step PCR. The first and second PCRs incorporated a 27-amino acid sequence representative of the 27 N-terminal codons of the OLE1 gene, which code for the endoplasmic reticulum (ER) localization sequence. Then, a ribosomal binding site and the recombination sites required for Gateway® cloning are introduced (FIG. 10A). By way of the pDONR221 entry clone, Invitrogen's Gateway® cloning technology incorporated each gene into the yeast expression plasmid pYES-DEST52 (FIGS. 10B,C). Sequencing of the entire gene is performed to ensure no mutations are introduced during the PCR reactions. The GAL1 promoter induces the expression of the gene in the presence of galactose and the 6×His tag allows for Western blot detection using a His-tag Monoclonal antibody kit available from Novagen.

In one example, to create a more stable variant of the SCD protein, one that is not easily degraded by an ER protease at the N-terminal, a truncated form of each mouse SCD isoform is created. PCR primers are designed to eliminate approximately 30 of the first amino acids corresponding to the protease site.

Each expression plasmid is transformed into the yeast strain L8-14C, an OLE1 deficient yeast mutant, according to the Saccharomyces cerevisiae EasyComp Transformation Kit (Invitrogen). Transformed cells are cultured on plates containing minimal medium (0.67% yeast nitrogen base w/o amino acids; 0.2% casamino acids; 2% Bacto™ agar) plus 0.005% histidine, 0.01% leucine, 2% D-glucose, and 0.5 mM UFAs. Cells are then selected from the plates and streaked on minimal medium plates containing 0.005% histidine, 0.01% leucine, and 2% D-galactose. Galactose induces expression of the inserted gene by acting on the GAL1 promoter region of the pYES-DEST52 plasmid.

Protein Expression

Time course expression trials are completed by first inoculating a 10 ml culture (synthetic culture medium without uracil {SC-U} containing 2% glucose and 0.5 mM UFAs) with a single isolated colony. The culture is grown overnight at 30° C. with agitation set at 280 rpm. After ˜30 hr of incubation the amount necessary to yield an OD₆₀₀ of 0.4 in a 50 ml culture is transferred to a clean culture tube. The cells are harvested by centrifugation at 3000×g for 10 minutes, resuspended in a small volume of SC-U medium containing 2% galactose (−UFAs), and this solution is used to inoculate a 50 mL of SC-U medium with 2% galactose. Cells are grown at 30° C. with agitation set at 280 rpm and samples are taken at 0, 4, 8, 12, 16, and 24 hours.

Cloning of all SCD enzymes is designed such that a His (6×)-tag is incorporated to facilitate detection of the expressed protein by Western and purification by affinity chromatography. To determine the optimal time for cell harvest after induction, samples are taken at different times and the expression of the SCD is assessed by Western blot.

Western Blot

Cells are lysed by vigorous vortexing in the presence of glass beads and samples run on a Tris-HCl gradient ReadyGel available from Bio-Rad (Hercules, Calif.). Western Blot analysis is used to visualize the SCD using a His-Tag® Monoclonal antibody that recognizes the 6×His tag and a goat anti-mouse IgG AP conjugate secondary antibody for detection with an alkaline phosphatase reagent (His-tag detection kit available from Novagen).

In Vivo Activity Assay

Full-length and truncated versions of each isoform, SCD1-SCD4, were successfully amplified and cloned. The use of Gateway® technology averaged >95% efficiency. Each SCD isoform is transformed into the OLE1 deficient yeast strain L8-14C. Transformed cells are capable of growing on minimal medium plates containing histidine, leucine, D-glucose, and UFAs, or on minimal medium plates containing histidine, and D-galactose, in the absence of unsaturated fatty acids. The results are summarized in Table 2. These experiments proved that that the enzymes are active in vivo. Note that the SCD4 enzyme appeared to differ from the other isoforms as the transformed yeast exhibited different growth patterns.

TABLE 2
Comparison of expression systems for the mammalian stearoyl-CoA
desaturases (4 mouse (m) isoforms, 2 human (h) isoforms, 1 and
mycobacterial (DesA3) isoform

| Enzyme | Protein constructs available | In vivo activity |
|---|---|---|
| mSCD1 | Full-length and truncate | ++++++ |
| mSCD2 | Full-length and truncate | ++++ |
| mSCD3 | Full-length and truncate | +++++ |
| mSCD4 | Full-length and truncate | Not active |
| hSCD1 | ole 1 chimera | +++ |
| hSCD5 | Wild-type | +++++ |
| DesA3 | Wild-type and ole 1 chimera | None without Rv32320c |

TABLE 3
Comparison of reconstituted complexes of recombinant mSCD1
(heterologous expression; prepared as yeast microsomes) with exogenous
recombinant cyt b5 and cyt b5 reductase (heterologous expression; prepared in
Escherichia coli)

| | | | | | | | mSCD1 + cyt b5 | | Complete | |
|---|---|---|---|---|---|---|---|---|---|---|
| | Background | | mSCD1 only | | mSCD1 + cyt b5 | | reductase | | complex | |
| mSCD1 | − | − | + | + | + | + | + | + | + | + |
| cyt b5 | − | − | − | − | + | + | − | − | + | + |
| cyt b5 | − | − | − | − | − | − | + | − | + | + |
| reductase | | | | | | | | | | |
| cpm 18:0- | 27,392 | 27,901 | 28,468 | 29,673 | 26,319 | 21,999 | 27,015 | 27,983 | 25,380 | 21,289 |
| CoA | | | | | | | | | | |
| cpm 18:1- | 569 | 562 | 1626 | 1743 | 1684 | 1761 | 1372 | 1513 | 1599 | 2018 |
| CoA | | | | | | | | | | |
| total cpm | 37,961 | 28,463 | 30,094 | 31,416 | 28,003 | 23,760 | 28,387 | 29,496 | 26,979 | 23,307 |
| % | 2.03% | 1.97% | 5.40% | 5.55% | 6.01% | 7.41% | 4.83% | 5.13% | 5.93% | 8.66% |
| conversion | | | | | | | | | | |
| average | 2.00% | | 5.48% | | 6.71% | | 4.98% | | 7.29% | |
| conversion | | | | | | | | | | |
| Correction by | 0.00% | | 3.47% | | 4.71% | | 2.98% | | 5.29% | |
| subtraction | | | | | | | | | | |
| of | | | | | | | | | | |
| background | | | | | | | | | | |
| % change | | | | | 35.64% | | −14.2% | | 52.35% | |
| vs. mSCD1 | | | | | | | | | | |

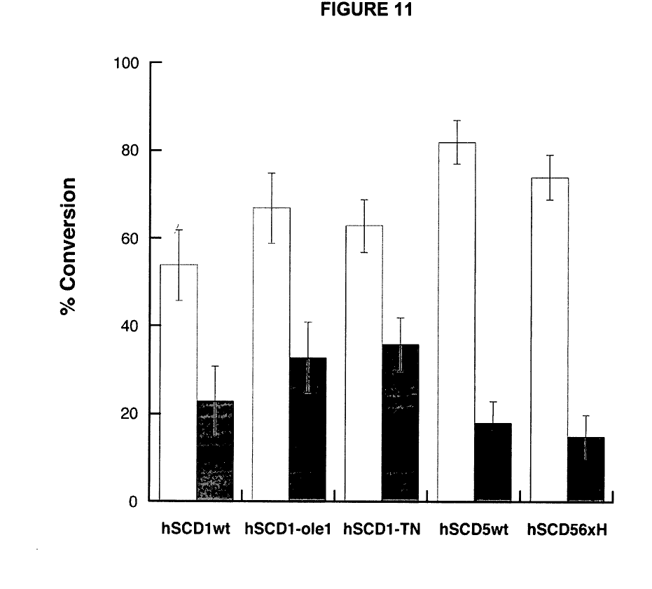

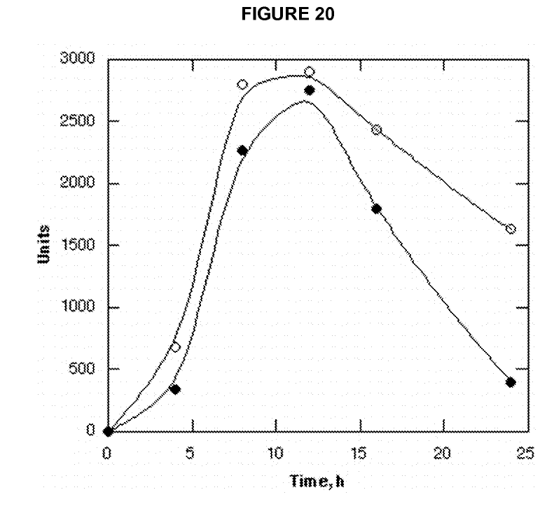

Representative results from the expression of full and truncated forms of SCD1 are shown in FIG. 11 and FIG. 20. The graph in FIG. 11 shows the distribution of unsaturated fatty acids derived from expression of human SCD1 and SCD5 in yeast. The graph in FIG. 20 is generated based on the band intensity from immunoprecipitation images of full-length SCD1 () and truncated SCD1 (∘). The optimal induction of SCD1 is between 8 and 12 hours. Cleavage of the full-length version of each isoform is observed. The truncated version of each isoform is more stable over longer periods of time.

Table 4 shows the fatty acid profile of transgenic L8-14C expressed as percentage of total C16 and C18 fatty acids detected.

TABLE 4
Fatty acid profile of transgenic L8-14C expressed as percentage of total
C16 and C18 fatty acids detected

| | | | | | Ratio |
|---|---|---|---|---|---|
| Enzyme | 16:0 | 16:1 | 18:0 | 18:1 | 18:1/16:1 |
| hSCD1-wt | 61.87 | 23.22 | 9.40 | 14.65 | 0.63 |
| hSCD1-ole1 | 39.31 | 24.15 | 9.68 | 26.84 | 1.08 |
| hSCD1-TN | 51.76 | 21.25 | 10.98 | 16.00 | 0.76 |
| hSCD5-wt | 45.032 | 8.90 | 7.73 | 30.20 | 3.39 |
| hSCD5-6xH | 56.64 | 6.70 | 10.79 | 25.70 | 3.73 |

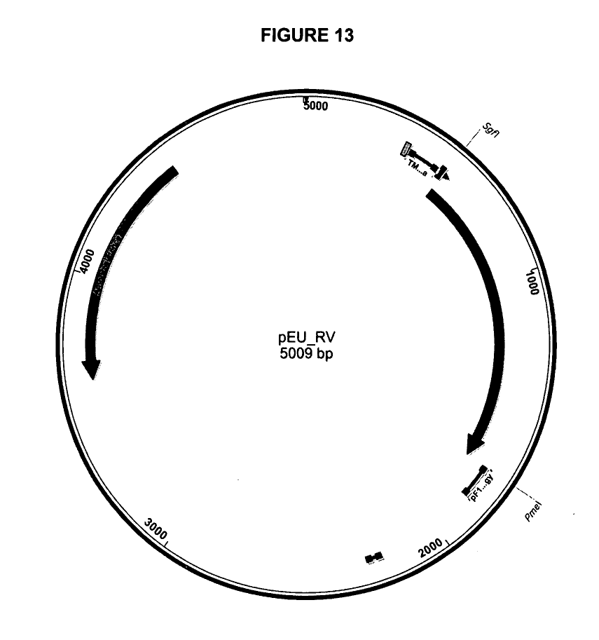

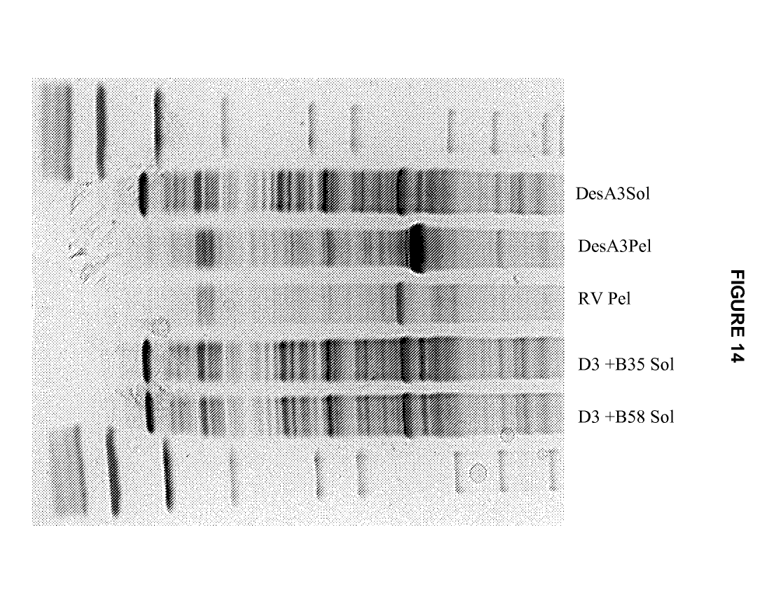

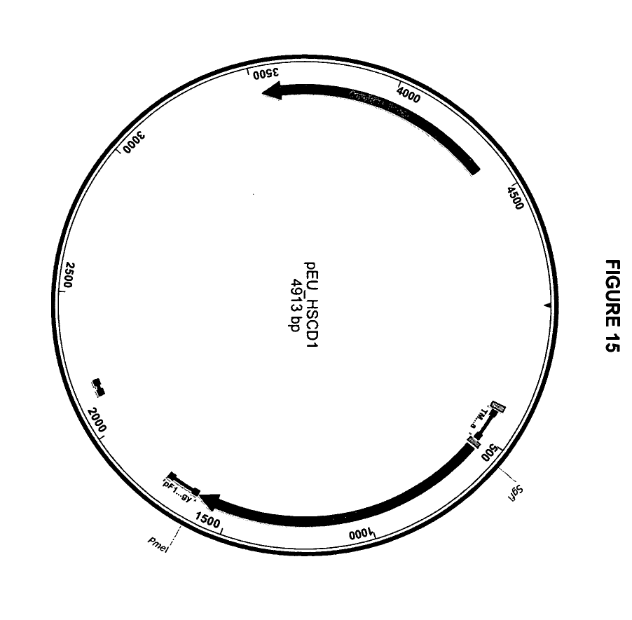

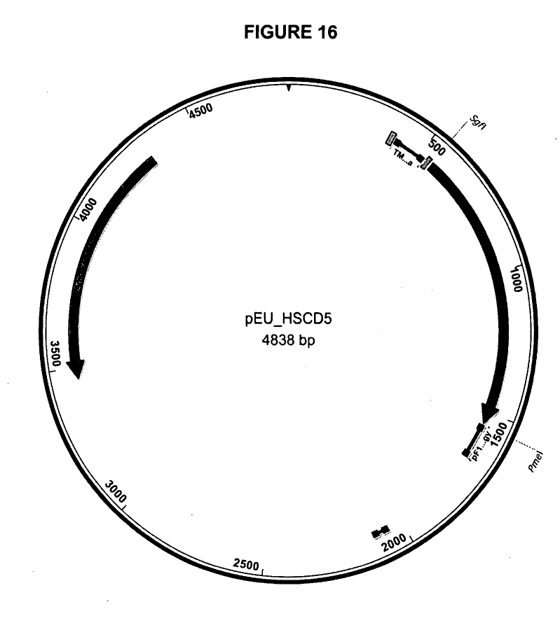

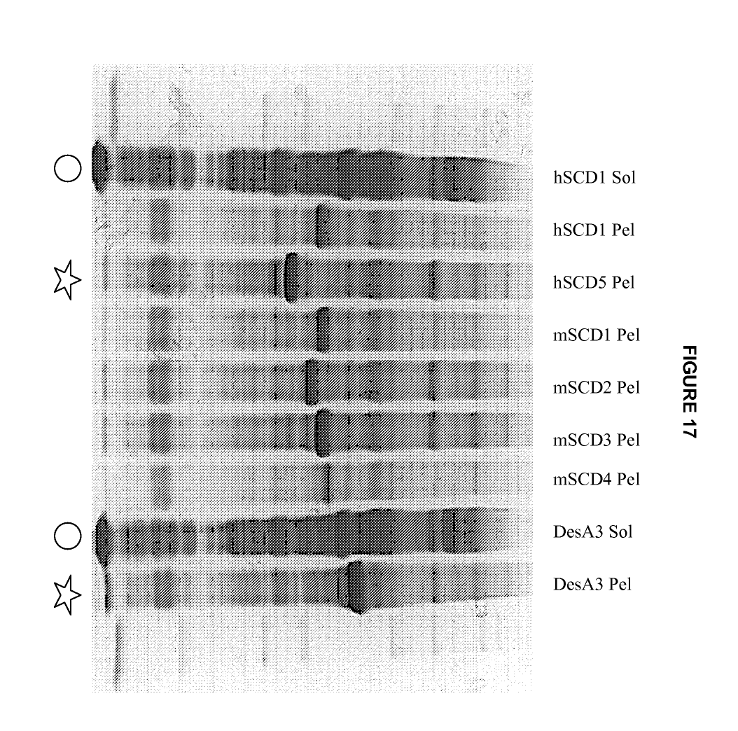

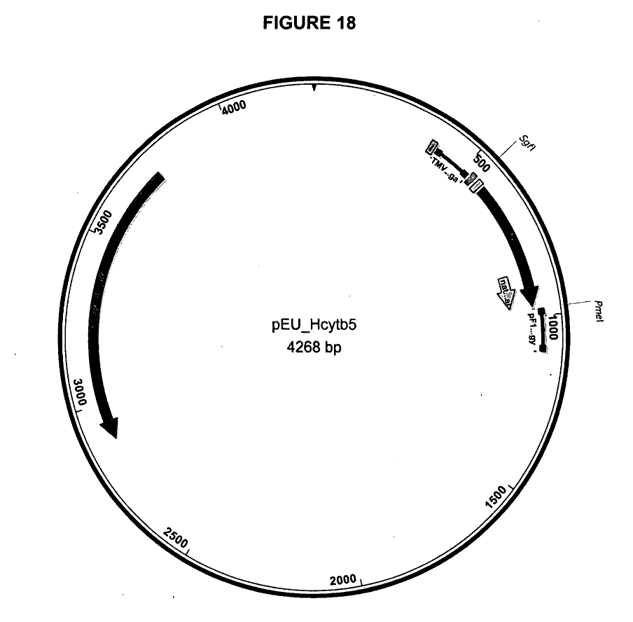

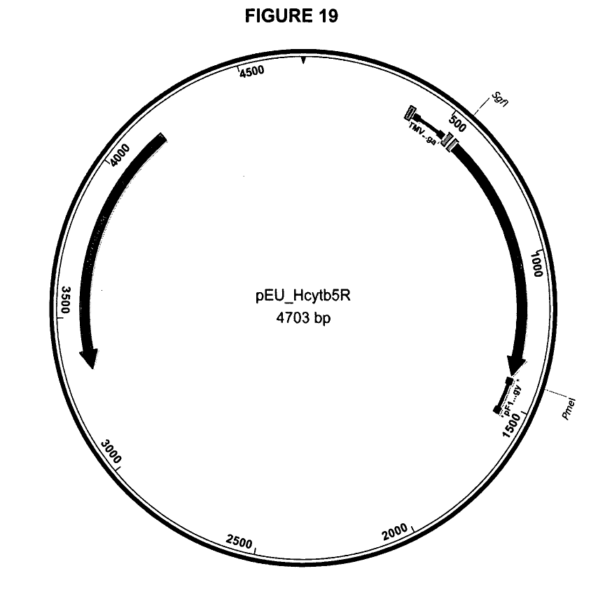

FIG. 14 and FIG. 17 show the results of expression of human, mouse and mycobacterial desaturases in wheat germ cell-free translation using vectors claimed in FIG. 12, FIG. 13, FIG. 15, FIG. 16, FIG. 18, and FIG. 19. As shown in FIG. 14, co-expression is achieved in cell-free systems by titrated addition of individual genes. In this example, specialized constructs are not needed.

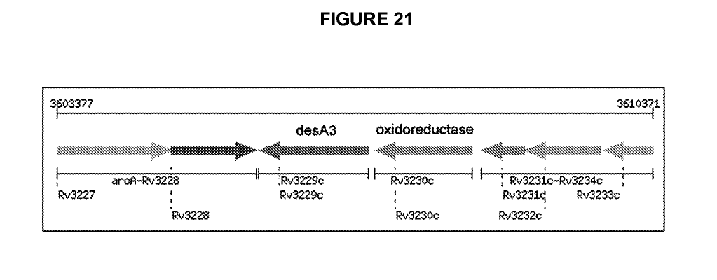

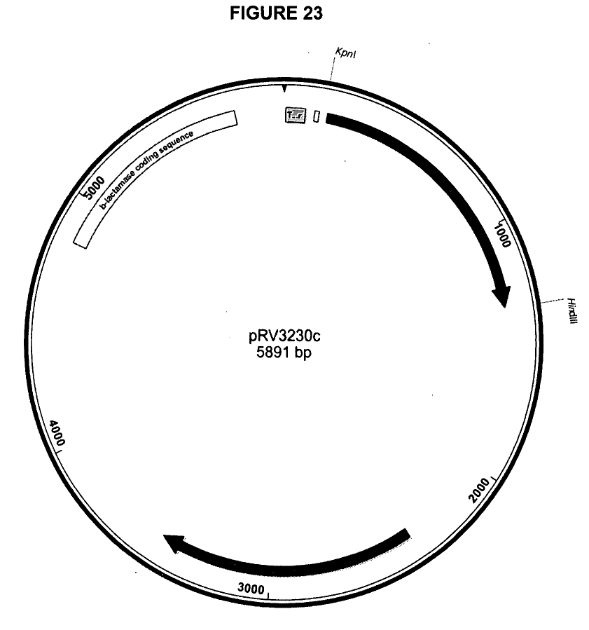

The broader applicability of the invention is shown in the following example, which analyzes expression and enzymatic activity the mycobacterial DesA3 operon and genes in its vicinity. FIG. 21 schematically depicts the genes in the vicinity of a mycobacterial DesA3 (i.e., Rv3229c), as obtained from http://www.doe-mbi.ucla.edu/TB/. According to expression studies using microarray work, DesA3 and Rv3230c of the DesA3 operon are coordinately expressed (http://www.doe-mbi.ucla.edu/-strong/map/; see also Betts et al. 2002, Mol. Microbiol. 43: 717-731). There is an adjacent conserved protein of interest, Rv3231c, whose function is not yet known, although it also may be a potential protein of the DesA3 operon. The inventors cloned, expressed and isolated both DesA3 (Rv3229c) and Rv3230c. The Rv3230c gene is annotated as a putative oxidoreductase in intermediary metabolism. The data presented here demonstrate a function for Rv3230c, as a previously unidentified oxidoreductase function, to the DesA3 complex. FIG. 22 shows an expression vector for constitutive expression of DesA3 in mycobacteria. FIG. 23 shows an expression vector for inducible expression of Rv3230c in Escherichia coli. FIG. 24 shows an expression vector for co-expression of DesA3 and Rv3230c in mycobacteria.

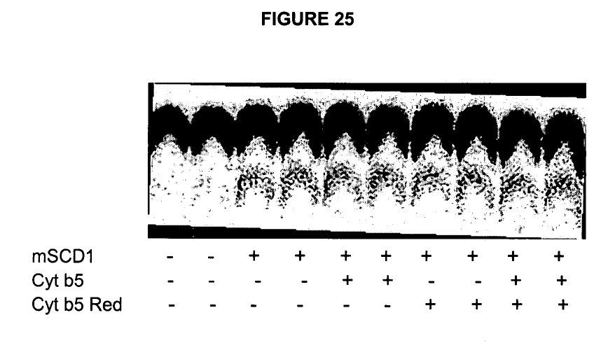

The efficacy of the expression system is shown in the following example. FIG. 25 shows a phosphorimager image (top) and quantitative analysis (bottom) of duplicate trials for the conversion of [¹⁴C]-18:0-CoA to [¹⁴C]-18:1-CoA by recombinant mouse SCD1 in the presence of various combinations of recombinant preparations of cytochrome b5 and cytochrome b5 reductase.

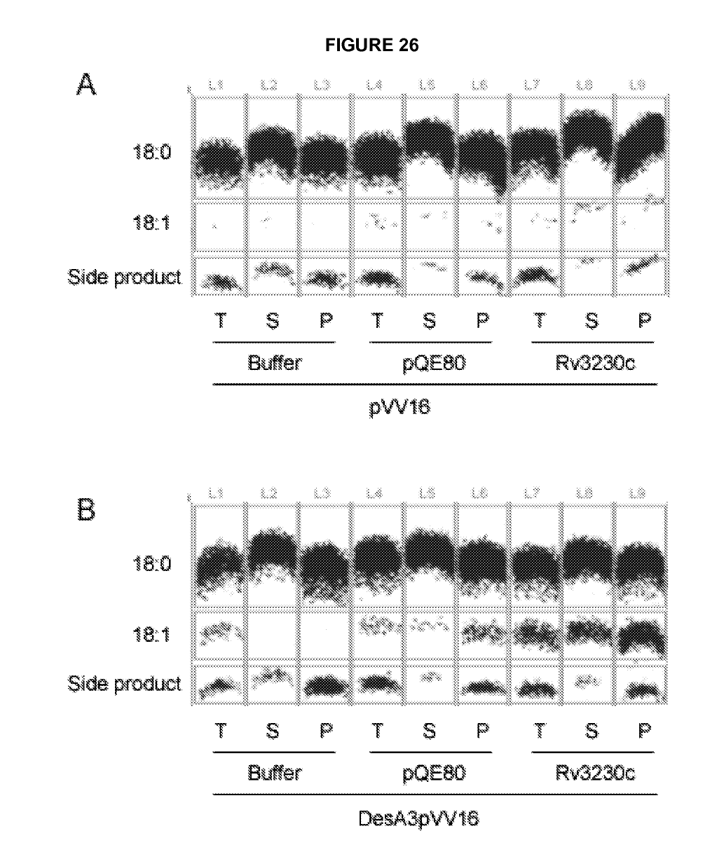

FIG. 26 shows activity assays after expression of DesA3 from vector DesA3HispVV16 in Mycobacterium smegmatis. The product 18:1-CoA is indicated. T, S, and P correspond to the total, supernatant, and pellet fractions of Mycobacterium smegmatis lysate, respectively. Shown in (A) are data obtained for the control—empty vector pVV16. The upper band is the unreacted substrate, the bottom band is the side reaction product from Mycobacterium smegmatis lysate, and the middle band is the oleic acid product, confirmed by standard reaction. Addition of Rv3230c (expressed and partially purified from Escherichia coli) gives a greater than 10-fold increase in the rate of production of 18:1-CoA. In different experiments where the amount of Rv3230c is varied, the increase in activity of production of 18:1-CoA is up to 30-fold, indicating the existence of a multi-protein complex for DesA3 activity in Mycobacterium tuberculosis. The decrease in activity beyond addition of the optimal amount of Rv3230c is assigned to excess consumption of the cosubstrate NADPH, which is consistent with unbalanced oxidoreductase activity relative to desaturase activity.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of genetic engineering, molecular biology, and biochemistry, are within the scope of this invention. All publications, patents, and patent applications cited herein are incorporated by reference in their entirety for all purposes.

What is claimed is:

1. A vector that expresses a desaturase system, which comprises:
one or more first genes encoding a desaturase; and
one or more second genes encoding an oxidoreductase,
wherein said first and second genes are operably linked to a promoter.

2. The vector of claim 1 wherein the first and second genes are each independently operably linked to a first and second promoter, respectively.

3. The vector of claim 1 wherein the desaturase is selected from the group of fatty acid desaturases capable of inserting double bonds into fatty acyl chains derivatized to CoA, glycerols, alkyl ethers, alkenyl ethers, phosphatides, mycolic acids, or glycosidic sugars.

4. The vector of claim 3 wherein the desaturase is a stearoyl-CoA desaturase.

5. The vector of claim 1 wherein the oxidoreductase is selected from the group consisting of oxidoreductases that are specific for NADH or NADPH, and that reduce enzyme-bound metal ions including heme groups, iron-sulfur centers and those bound by amino acid side chains such as histidine, glutamate, aspartate, cysteine, or tyrosine.

6. The vector of claim 5 wherein the oxidoreductase is a cytochrome b5.

7. The vector of claim 5 wherein the oxidoreductase is a cytochrome b5 reductase.

8. The vector of claim 5 wherein the oxidoreductase is Rv3230c.

9. The vector of claim 1 wherein the first gene encodes stearoyl-CoA desaturase, and the second genes encode cytochrome b5 and cytochrome b5 reductase.

10. The vector of claim 1 wherein at least one gene is operably linked to a ribosomal binding site sequence.

11. The vector of claim 1 wherein each gene encoding a desaturase or an oxidoreductase is operably linked to a ribosomal binding site sequence.

12. The vector of claim 1 wherein the desaturase system comprises a fusion protein.

13. The vector of claim 12 wherein the fusion protein comprises stearoyl-CoA desaturase and cytochrome b5.

14. The vector of claim 12 wherein the fusion protein comprises stearoyl-CoA desaturase and cytochrome b5 reductase.

15. The vector of claim 1 wherein at least one gene is operably linked to a tag sequence.

16. The vector of claim 15 wherein the tag sequence encodes polyhistidine.

17. The vector of claim 1 wherein the vector is selected from the group consisting of plasmids, phages, phagemids, viruses, and artificial chromosomes.

18. A method for assaying the activity of a desaturase, which comprises:
a) expressing one or more first genes encoding a desaturase;
b) expressing one or more second genes encoding an oxidoreductase;
c) contacting the expressed desaturase and oxidoreductase with a fatty acid; and
d) determining the increase in activity of 18:1-CoA production.

19. The method of claim 18 wherein the desaturase is a stearoyl-CoA desaturase.

20. The method of claim 18 wherein the oxidoreductase is a cytochrome b5.

21. The method of claim 18 wherein the oxidoreductase is a cytochrome b5 reductase.

22. The method of claim 18 wherein the oxidoreductase is Rv3230c.
